(19) United States
(12) Patent Application Publication
(10) Pub. No.: US 2023/0374561 A1
(43) Pub. Date: Nov. 23, 2023

(54) EXTRACTION OF PSILOCYBIN AND PSILOCIN

(71) Applicant: CHEMTOR, LP, Lockhart, TX (US)

(72) Inventors: William LANIER, Lake Placid, FL (US); Shane Jacob Hamill BENJAMIN, Simla, CO (US)

(21) Appl. No.: 18/327,946

(22) Filed: Jun. 2, 2023

Related U.S. Application Data
(63) Continuation of application No. PCT/US2023/022720, filed on May 18, 2023.
(60) Provisional application No. 63/364,927, filed on May 18, 2022.

(51) Int. Cl. C12P 17/10; C12N 9/42; B01D 11/02

(52) U.S. Cl. CPC C12P 17/10; C12N 9/2442; C12Y 302/01014; B01D 11/0288

(57) ABSTRACT

A method of extracting and isolating psilocybin and/or psilocin from psilocybin fungi includes forming a mixture of the fungi, an extraction solution, and a chitinase capable of digesting chitin present in the fungi. Solids can be filtered from the mixture to yield an extract including psilocybin and/or psilocin. The extract can be purified to isolate the psilocybin and/or psilocin.

### CROSS REFERENCE TO RELATED APPLICATIONS

The present application is a continuation of PCT International Patent Application No. PCT/US2023/022720 filed May 18, 2023, and entitled “EXTRACTION OF PSILOCYBIN AND PSILOCIN,” which claims benefit of priority to U.S. Provisional Patent Application No. 63/364,927 filed May 18, 2022, entitled “EXTRACTION OF PSILOCYBIN”, the disclosures of which are hereby incorporated by reference in their entirety.

### FIELD OF DISCLOSURE

The present disclosure is related to purification and extraction of psilocybin and psilocin.

### BACKGROUND

Most or nearly all fungi of the genus Psilocybe contain psychedelic compounds psilocybin, psilocin, and baeocystin (“psilocybin mushrooms”). Psilocybin is a phosphorylated counterpart of psilocin, which is a pharmacologically active agent that acts on serotonin receptors in the brain, and psilocybin is rapidly dephosphorylated in the body to psilocin. Psilocybin (3-[2-(dimethylamino)ethyl]-1H-indol-4-yl dihydrogen phosphate) has the following chemical formula:

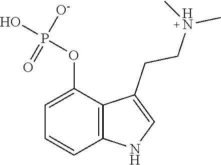

Psilocybin is of pharmacological importance with known uses in treating drug dependence, anxiety, and mood and mental health disorders, such as post-traumatic stress disorder (PTSD) and depression. There is also evidence that psilocybin can induce neural regeneration and/or increase neuroplasticity.

Psilocybin can be synthetically produced, but methods for producing synthetic psilocybin are expensive. As such, there is a demand for psilocybin extracts from natural sources. Psilocybin and psilocin may be extracted and isolated from caps, stems, and/or spores of psilocybin mushrooms. However, current extraction methods, such as maceration and Soxhlet extraction, have very low efficiency, often 15% or less, and may be highly time-consuming and/or labor-intensive. This is due, at least in part, to the variable nature of the psilocybin mushrooms (crop-to-crop variability) as well as the chemical characteristics of psilocybin, psilocin, and the other compounds present in the mushrooms, such as chitin. Accordingly, there remains a need for a simplified, efficient extraction method for isolating psilocybin and psilocin from psilocybin mushrooms.

### BRIEF DESCRIPTION OF THE DRAWINGS

The following FIGURES illustrate embodiments of the subject matter disclosed herein. The claimed subject matter may be understood by reference to the following description taken in conjunction with the accompanying FIGURES, in which:

FIG. 1 is a diagram of an extraction system according to embodiments of the present disclosure.

FIG. 2 is a schematic of an extraction process according to embodiments of the present disclosure.

### DETAILED DESCRIPTION

The following disclosure provides many different embodiments or examples. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

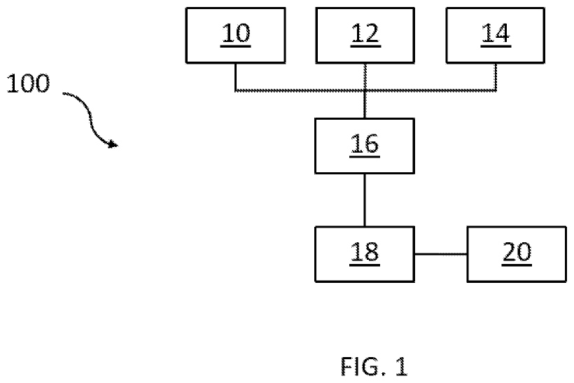

Referring to FIG. 1, a system 100 for extraction and isolation of psilocybin and psilocin from psilocybin mushrooms (referred to herein as “fungi”) is shown. The system 100 includes fungi supply 10 configured to supply fungi to an extraction vessel 16. The fungi supply 10 may include one or more species of fungi. In some embodiments, the fungi include one or more species from the genus Psilocybe, such as Psilocybe cubensis (Stropharia cubensis), Psilocybe cyanescens, Psilocybe semilanceata, and/or Psilocybe azurescens. The fungi in the fungi supply 10 may be powdered or ground. In some embodiments, the fungi supply 10 includes a grinder.

Chitin is a component of the cell walls of fungi, such as psilocybin mushrooms. Chitin is a stable molecule, resistant to degradation, and insoluble in typical solvents such as water, organic solvents, and mild acidic or basic solutions. The presence of chitin therefore may negatively affect extraction efficiency of psilocybin. In order to overcome this issue, chitin is digested in the present extraction system 100.

Chitinases are hydrolytic enzymes that break down glycosidic bonds in chitin. Chitinases are naturally found in organisms that either need to reshape their own chitin or dissolve and digest the chitin of fungi or animals. For example, fungi may produce their own chitinases to, for example, modify or remodel cell walls or aid in cell division. Naturally derived and synthetic chitinases are commercially available. As such, it is believed that enzymes derived from members of the Psilocybe genus and/or those taxa that naturally defend against these fungi in the wild, will likely prove to be the most effective in terms of breaking down classes of chitin specific to these fungi. An example of a suitable chitinase is that derived from Aspergillus niger.

The system 100 includes an enzyme supply 12 configured to supply chitinase to the extraction vessel 16. The enzyme supply 12 may include one or more chitinases. In some embodiments, the chitinase may be specified based on the fungi present in fungi supply 10. In some embodiments, the enzyme supply 12 may include an agitator, such as a stir bar.

The system 100 further includes an extractant supply 14 configured to supply an extraction solution to the extraction vessel 16. In some embodiments, the extraction solution is water. In some embodiments, the extraction solution is pH adjusted to pH 5-8. In other embodiments, the temperature is adjusted to 30-40° C. In some embodiments, the extraction solution includes one or more salts dissolved therein. In some embodiments, the extraction solution is ethanol or a mixture of water and ethanol. In some embodiments, the extraction solution may include acetone, chloroform, ether (e.g., dimethyl ether or diethyl ether), hexane, methanol, water, or combinations thereof. In some embodiments, the extraction solution includes more than one of the foregoing due to the different solubilities of psilocybin and psilocin. That is, psilocybin is soluble in water, slightly soluble in acetone, ether, and methanol, and insoluble in chloroform and hexane. Conversely, psilocin is soluble in acetone, chloroform, ether, hexane, and methanol, and slightly soluble in water.

The extraction vessel 16 is configured to receive ground fungi from the fungi supply 10, the extraction solution from extractant supply 14, and the chitinase from the enzyme supply 12. In some embodiments, the extraction vessel 16 may receive the fungi, extraction solution, and chitinase in a weight ratio of at least 0.1 g, 1 to 100 g, 5 to 80 g, 10 to 60 g, or 20 to 40 g of chitinase per g of fungi digested per each hour. Increasing the number of units of chitinase per gram will result in shorter digestion times. In some embodiments, a weight ratio of chitinase to fungi is 0.1 to 10, 0.1 to 1, 0.1 to 2, 0.2 to 5, 0.3 to 2, about 0.5, about 1, or about 2:3.

The extraction vessel 16 may include an agitator, such as a stir bar or air agitator to enhance extraction efficiency. In some embodiments, the extraction vessel 16 first receives the fungi and extraction solution, mixes the fungi and extraction solution, and then the chitinase is supplied 1 minute, 5 minutes, or 15 minutes thereafter. In other embodiments, the components may be supplied simultaneously. In yet other embodiments, the extraction solution and the chitinase may be first mixed in the extraction vessel and then the fungi may be supplied immediately thereafter.

In some embodiments, the extraction vessel 16 includes a heater, wherein the extraction vessel may be maintained at an elevated temperature to facilitate digestion of chitin in the fungi by the chitinase. In some embodiments, the extraction vessel 16 is configured to be maintained at a temperature of from 25 to 75° C. with higher temperatures being feasible if chitinase from extremophiles are utilized.

In operation of the system 100, the extraction mixture (fungi, extraction solution, and chitinase) has a set residence time in the extraction vessel 16. During the residence time, the extraction mixture may be continuously mixed, intermittently mixed, or not mixed. During the residence time, the extraction mixture may be maintained at a single temperature or the temperature may vary. In some embodiments, the residence time is at least 1 hour, at least 6 hours, at least 12 hours, at least 24 hours, about 12 hours, or about 24 hours relative to the ratio of fungi present to units of chitinase added.

After the residence time has expired, the extraction mixture is transferred from the extraction vessel 16 to a purification process 18. The purification process 18 is configured to isolate the psilocybin from remaining components in the extraction mixture. In some embodiments, the purification process 18 may include a filter as an initial component, wherein the filter may remove any remaining solids from the extraction mixture prior to purification. In other embodiments, the extraction mixture is separated from solids using a centrifuge. In some embodiments, the extraction mixture does not include any solid matter (i.e., the extraction may fully digest the fungi).

In some embodiments, the purification process 18 includes a conduit reactor, such as those described in U.S. Pat. No. 11,198,107, which is hereby incorporated by reference in its entirety. In such embodiments, the purification process 18 includes contacting the extraction mixture with a solvent in the conduit reactor. The solvent is immiscible (or only slightly miscible) with water and psilocybin and/or psilocin is more soluble in the solvent than in the extraction solution of the extraction mixture. In some embodiments, the solvent is ethyl acetate. Ethyl acetate is a polar-nonpolar solvent that is slightly miscible with water.

The contact between the aqueous extraction mixture and the solvent transfers psilocybin from the extraction mixture into the solvent while leaving one or more unwanted components in the extraction mixture. In some embodiments, an additive may be added to the solvent and/or extraction mixture to aid in transferring the psilocybin into the solvent. For example, in some embodiments, a phase transfer catalyst may be used. In some embodiments, the contacting may be facilitated by simultaneously introducing the extraction mixture, solvent, and, optionally, the additive(s) into a first end of the conduit reactor, wherein the reaction products may then be received in separator opposite the first end. In such embodiments, the reaction products include a heavier aqueous phase comprising the extraction solution and a light organic phase comprising the solvent and psilocybin. The two phases may then be separately removed from the separator. The organic phase (a psilocybin/psilocin isolate) may be further refined to remove additional unwanted components or may constitute a final product. The aqueous phase may be discarded, remediated, or further processed to extract desirable compounds therefrom (e.g., residual psilocybin/psilocin). In some embodiments, the contacting may involve mixing the extraction mixture, solvent, and, optionally, the additive(s) in a vessel and then introducing this mixture into the first end of the conduit reactor. The resulting reaction products may be as described above.

In some embodiments, the purification process 18 utilizes a salting out technique to isolate the psilocybin and/or psilocin from the extraction mixture. In such embodiments, a buffered solution of phosphate salt, such as sodium phosphate, may be added to the extraction mixture to or beyond a saturation point. As a result, phosphate-containing compounds, such as psilocybin fall out of solution in solid form. The solid psilocybin and/or psilocin may then be isolated using, e.g., filtration or dialysis to yield a psilocybin isolate which may be further purified through, e.g., recrystallization.

In some embodiments, the purification process 18 utilizes a chromatography column to isolate the psilocybin and/or psilocin from the extraction mixture. In such embodiments, suitable solvents may include water, acetone, DMSO, methanol, ethanol, and/or a mixture thereof.

After the extraction mixture has been purified in the purification process 18, the resulting psilocybin and/or psilocin isolate may be transferred to an isolate tank 20.

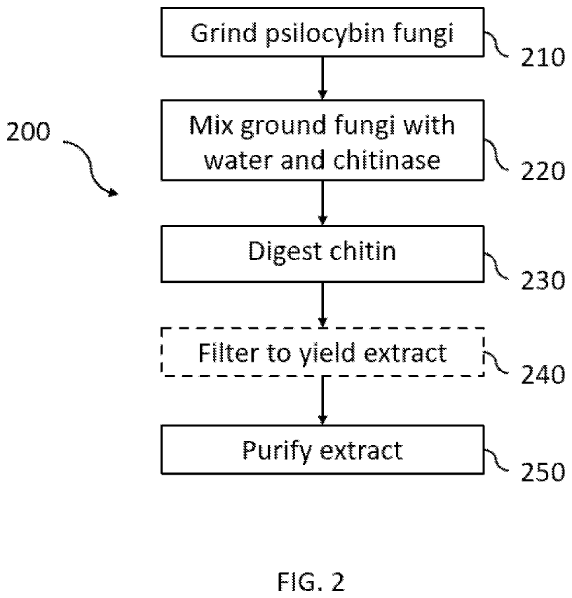

Turning to FIG. 2, a method 200 of extracting and isolating psilocybin and/or psilocin from fungi is depicted. In step 210 of the method 200, the fungi is ground into a powder. The grinding step 210 may be conducted prior to introducing the fungi into the fungi supply 10 or may be conducted in the fungi supply 10. Next, in step 220, the fungi, extraction solution, and chitinase are supplied to the extraction vessel 18 from the fungi supply 10, extractant supply 14, and enzyme supply 12, respectively, and mixed. The mixing may be facilitated by an agitator disposed in the extraction vessel 18. In step 230, the chitinase digests the chitin of the fungi. Step 230 may begin during step 220 or may entirely coincide with step 220. In some embodiments, steps 220 and/or 230 involve heating the mixture as described above. In some embodiments, the method 200 includes a step 240 of filtering solids from the mixture to yield an extract. In other embodiments, steps 220 and 230 yield the extract. That is, in some embodiments, steps 220 and 230 may entirely liquify the mixture such that the filtering step 240 is not required. Lastly, the method 200 includes a step 250 of purifying the extract. Step 250 may utilize the extraction process 18 described above. Step 250 yields a psilocybin and/or psilocin isolate.

According to embodiments of the present disclosure, extraction efficiency of psilocybin and/or psilocin from psilocybin mushrooms may be greatly improved. This is due at least in part to the digestion of chitin using one or more chitinases, which allows for more effective extraction of psilocybin into the extraction solution.

### Examples

Five sample sets were prepared, each containing 15 grams of Psilocybe cubensis biomass. The extraction solvents used across these samples were water, ethanol, or a combination of both; with or without the addition of chitinase. The specific compositions of the sample sets were as follows:
Comparative Sample #1 used 250 grams of ethanol as the solvent;
Sample #2 employed 10 grams of chitinase and 250 grams of ethanol;
Sample #3 contained 10 grams of chitinase, 125 grams of water, and 125 grams of ethanol;
Sample #4 utilized 10 grams of chitinase and 250 grams of water;
Reference Sample #5 was 15 grams of Psilocybe cubensis used as a reference.

Each sample underwent extraction in the above-referenced extractant solvents for 24 hours before undergoing sonication at 250 watts and 50% amplitude for 3 minutes. Immediately following sonication, each sample was subjected to gravity filtration for 10 minutes using a 20 micrometer filter paper and a gravity filtration system. Each extractant was collected and tested using gas chromatography and mass spectroscopy (GCMS). Table 1 below summarizes the results.

TABLE 1

| Sample | Comp. 1 | 2 | 3 | 4 | Ref. 5 |
|---|---|---|---|---|---|
| Solvent (g) | 250 ethanol | 250 ethanol | 125 ethanol/ | 250 water | N/A |
| | | | 125 water | | |
| Chitinase (g) | 0 | 10 | 10 | 10 | N/A |
| Analyte 1: Psilocin (μg/gm) | 8.5 | 6.3 | 16.3 | ND | 146 |
| Analyte 2: Psilocybin (μg/gm) | 57.0 | 27.7 | 1.5 | ND | 8395 |
| Recovered Solvent (g) | 79 | 191 | 112 | 167 | N/A |
| Total Conc. Analyte 1 (μg) | 671.5 | 1203.3 | 1825.6 | ND | 2190 |
| Total Conc. Analyte 2 (μg) | 4503 | 5289.7 | 168 | ND | 125925 |
| % Recovery Analyte 1 | 30.7 | 55.0 | 83.4 | 0 | N/A |
| % Recovery Analyte 2 | 3.6 | 4.2 | 0.1 | 0 | N/A |

“ND” in Table 1 above indicates that the value was below the detectable level of the instrumentation.

The results in Table 1 of psilocin and psilocybin recovered were determined for each sample post-filtration of extracted biomass and calculated by multiplying the concentration of each analyte as determined via GCMS by the total mass of extractant recovered. The percent recovery of each analyte was then determined by comparing the total yield recovered to the Reference Sample #5. This is applicable because each 15 gram sample, as well as the reference, is a subset of a larger homogenized pool of mushrooms that was divided into each sample post homogenization.

As seen in Table 1, without the use of chitinase, Comparative Sample #1 was only able to recover 30.7% of psilocin and 3.6% of psilocybin, wherein Sample #2, using the same solvent with the addition of chitinase, was able to include the recovery amounts to 55.0% and 4.2%, respectively.

Although various embodiments have been shown and described, the disclosure is not limited to such embodiments and will be understood to include all modifications and variations as would be apparent to one of ordinary skill in the art. Therefore, it should be understood that the disclosure is not intended to be limited to the particular forms disclosed; rather, the intention is to cover all modifications, equivalents, and alternatives falling within the spirit and scope of the disclosure as defined by the appended claims.

What is claimed is:

1. A method comprising:
forming a mixture of
a fungus comprising chitin and psilocybin or psilocin;
an extraction solution; and
a chitinase capable of digesting the chitin;
separating solids from the mixture to yield an extract comprising the psilocybin and/or the psilocin.

2. The method of claim 1, further comprising purifying the extract to isolate the psilocybin and/or the psilocin.

3. The method of claim 2, wherein purifying comprises
contacting the extract with a solvent that is immiscible with the extraction solution, wherein psilocybin and/or psilocin is more soluble in the solvent than in the extraction solution; and
separating the solvent comprising the psilocybin and/or psilocin from the extract.

4. The method of claim 1, further comprising grinding the fungus prior to forming the mixture.

5. The method of claim 1, further comprising heating the mixture to a temperature of 25° C. to 100° C. and maintaining the mixture at said temperature for at least 6 hours prior to the separating step.

6. The method of claim 2, wherein purifying comprises chromatography.

7. The method of claim 2, wherein the extract comprises the psilocybin and purifying comprises adding a phosphate salt to the extract until psilocybin falls out of the mixture and separating the psilocybin from the mixture.

8. The method of claim 1, wherein the extraction solution comprises acetone, chloroform, dimethyl ether, diethyl ether, hexane, methanol, water, or combinations thereof.

9. The method of claim 3, wherein the extraction solution comprises acetone, chloroform, dimethyl ether, diethyl ether, hexane, methanol, water, or combinations thereof; and wherein the solvent comprises ethyl acetate.

10. The method of claim 1, wherein a weight ratio of the chitinase to the fungus in the mixture is from 0.1 to 2.

11. The method of claim 1, wherein forming the mixture comprises agitating the fungus, the extraction solution, and the chitinase for 6 to 24 hours before the separating step.

12. A system comprising:
an extraction vessel;
a fungus supply comprising a fungus and configured to supply the fungus to the extraction vessel, wherein the fungus comprises chitin and psilocybin or psilocin;
an extractant supply comprising an extraction solution and configured to supply the extraction solution to the extraction vessel; and
an enzyme supply comprising a chitinase and configured to supply the chitinase to the extraction vessel, wherein the chitinase is capable of digesting the chitin.

13. The system of claim 12, wherein the extraction vessel comprises a heater and an agitator.

14. The system of claim 12, further comprising a purifier configured to receive an aqueous extract from the extraction vessel and form an isolate of the psilocybin or the psilocin.

15. The system of claim 14, wherein the purifier comprises a chromatography column.

16. The system of claim 14, wherein the purifier comprises a conduit reactor having a plurality of fibers disposed therein;
wherein the extraction vessel is configured to supply the extract to a first end of the conduit reactor; and
wherein the system further comprises
a solvent supply comprising a solvent and configured to supply the solvent to the first end of the conduit reactor, wherein the solvent is immiscible with the extraction solution, wherein psilocybin and/or psilocin is more soluble in the solvent than in the extraction solution; and
a separator disposed opposite the first end of the conduit reactor and configured to receive reaction products, wherein the reaction products comprise an aqueous phase comprising the extraction solution and an organic phase comprising the solvent and psilocybin and/or psilocin.

17. The system of claim 12, wherein the fungus supply comprises a grinder.

18. The system of claim 14, further comprising a filter positioned between the extraction vessel and the purifier.

19. The system of claim 12, wherein the extraction solution comprises acetone, chloroform, dimethyl ether, diethyl ether, hexane, methanol, water, or combinations thereof.

20. The system of claim 16, wherein the extraction solution comprises acetone, chloroform, dimethyl ether, diethyl ether, hexane, methanol, water, or combinations thereof; and wherein the solvent comprises ethyl acetate.
